(19) United States
(12) Patent Application Publication
(10) Pub. No.: US 2004/0245085 A1
(43) Pub. Date: Dec. 9, 2004

(54) Process and synthesizer for molecular engineering and synthesis of materials

(76) Inventor: Gopalakrishnan Srinivasan, Chennai (IN)

Correspondence Address: David Guerra, Suite 3, 933 Oleander Way South, South Pasadena, CA, 33707, US

(21) Appl. No.: 10/494,453

(22) PCT Filed: Aug. 7, 2002
(86) PCT No.: PCT/IN02/00165

(30) Foreign Application Priority Data
Mar. 13, 2002 (IN) 174/MAS/2002

(51) Int. Cl. C07C 1/00; C07C 2/00; C07C 4/00

(52) U.S. Cl. 204/157.15; 422/186.01

(57) ABSTRACT

A novel process of molecular engineering of materials for synthesis using electron excitations and a synthesizer capable of carrying out the process of electron excitations effectively is given in the specification. The synthesizer changes the properties of the material and also act as an electronic catalytic convertor or a fluid or fuel reformulator or as a heat pump for altering the kinetics of chemical reactions performance of internal combustion engines, heat engine cycles and finds wide use in multivarious applications including emission control, clean energy production, synthesize of new products, generation of LASER beam using carbondioxide in the automobile exhaust water treatment plants, refrigeration machines. Communications, etc.

### TECHNICAL FIELD

This invention relates to molecular engineering of the materials enabling control of synthesis, catalysis combustion, emissions from combustion and heat engine cycle performance. This invention is also directed to a novel process and construction of a synthesizer for molecular engineering of the fluids and chemicals to control catalysis combustion, emissions from combustion and the performance of any heat engine cycle or chemical process.

### BACKGROUND AND PRIOR ART

The physical property, chemical property, charge transfer and the state of an element or a compound, molecular structure, bond orientations, bond strength prior to a chemical reaction determines the chemical kinetics, the end products, its yield in a chemical process and the efficiency of operation of a heat engine cycle or a machine for a given energy input. By changing the properties, the state of an element or a compound through charge transfer, molecular engineering prior to a chemical reaction process commencement or during a process in a machine, it is possible to control the chemical kinetics namely the rate constant, reaction time, reaction rate the end products, it's yield mass balance, heat transfer and also enhance the performance or efficiency of a machine or heat engine cycle using such element or the compound for the energy exchange.

Catalysis and synthesis are achieved at present by making use of chemicals or elements, which do not directly participate in the reactions. The chemicals or the elements influence oxidation, reduction, acceleration, deceleration, mass balance of the process at low temperatures and pressures initializing the process from unexcited conditions Similarly, the process of combustion of fuels, heat transfer by liquids, gases or materials and most of the processes are initiated only from unexcited states of their molecules. At present, no single process or a single device is known to benefit, control or influence catalysis, synthesis, combustion, heat transfer by fluids, heat engine cycles performance, refrigeration cycle operation, vehicular emissions, internal combustion engine performance, chemical processes, change of physical properties and state of material, fuel or materials reformulation or rearrangement of molecular structure, synthesis for Laser beam generation, synthesis for hydrogen production, fuel cells efficiency enhancement, electricity production, communication, benefits of heat pump with multi various applications and uses at economical cost. Combustion emissions are mostly controlled by post treatment of the wasted exhaust with an oxidation or reduction catalysts or combination of both. No single process or single device is known to pre treat all the fuels for enabling clean combustion by exciting the molecules and by changing the kinetics of combustion for reduced emissions without using a chemical, noble metal catalyst or an additive. No process is known wherein catalysis is effected without the use of a chemical or an element as the catalyst. No single process or a single device is known for doing all the work namely to heat the refrigerant, fuels, fluid or materials during flow and also for changing their physical properties for better control, changing kinetics of the process for improved efficiency. No process or device is known to make use of the abundantly and continuously generated carbon dioxide, carbon monoxide, oxides of nitrogen gases from the automobile exhaust for the synthesis and making it suitable for the generation of laser beam which could be used for better navigation, collision prevention, pre treating the fuels and laser beam ignition in the engine cylinders for precision flame travel and combustion control. No process are device is known for use as a common catalytic converter for all the liquid and gaseous fuels and also for producing hydrogen rich fuels or hydrogen from materials that stores or contain hydrogen and generate electric current potential through ionization and ion liberation

The present invention is addressed to the aforesaid objective and has a technological answer to this goal with its novel process of molecular engineering of materials achieved through a synthesizer.

### OBJECTS OF THE INVENTION

The primary object of the present invention is to provide a process for molecular engineering which change any one or all of the following namely the physical property, chemical property, charges carried by the electrons or atoms or molecules or state of the element or the compound prior to a chemical reaction commencement for changing the chemical kinetics, the end products, it's yield, mass balance and heat release in a chemical process or the efficiency of operation of a heat engine cycle or a machine for a given energy input.

Another object of the present invention is to invent a contrivance, which facilitates the process of molecular engineering to take place and results in any one or all of the following namely the physical property change, chemical property change, transfer of charges, change in the state of an element or a compound prior to a chemical reaction commencement for changing the chemical kinetics, the end products it's yield, mass balance and heat release in a chemical process or the efficiency of operation of a heat engine cycle or a machine for a given energy input.

Yet another object of the invention is to provide a device for carrying out the novel process of molecular engineering of materials that acts as a fluid or fuel reformulator effecting any one or all of the following namely production of pieco second particles or electrons or atoms or molecules of meta stable states capable of transferring energy from the electron gas of the discharge plasma to the molecules to be activated through electron capture process or electron injection process or through resonant capture process for energy catalysis in addition to conventional surface catalysis or materials with rearranged molecular bond angles or molecular structure or materials of new formulation under excited conditions or material with altered viscosity or density or material with altered temperature or a combination of all above that could be used to advantage.

Further object of the present invention is to invent a contrivance, which facilitates the molecular engineering process to take place on the materials to change the kinetics of the combustion for reduced exhaust emission in automobiles, internal combustion engines and combustion equipments.

Yet another object of the present invention is to invent a device, which facilitates the molecular engineering process to take place for betterment of combustion resulting in reduced fuel consumption and performance improvements of the vehicle, internal combustion engines and other combustion equipments.

Another object of the of the present invention is to provide a device, wherein the device of the present works on leaded petrol, unleaded petrol Diesel, all fluids, gases and solids when suitable material of construction for the material flow passage is chosen.

Further object of the present invention is to provide a device to carry out the novel process of molecular engineering when fitted to the exhaust line of the automobiles or internal combustion engines or any exhaust gas lines in a combustion process excites the carbon di oxide, carbon monoxide and oxides of nitrogen molecules to make it suitable for synthesis and generation of laser (light amplification by stimulated emission radiation) beam, which could be used advantageously for several purposes.

Another object of the present invention is to provide a device to carry out the novel process of molecular engineering of the materials when fitted in the gas or liquid flow line of the refrigeration machines or any heat engine cycle or in any chemical process line enhances the performance and efficiency of the system due to any one or all of the following namely the changes in the properties of the material handled by it or by virtue of the device acting also as a heat pump adding energy input to the system.

Yet another object of the present invention is to provide a device to carry out the novel process of molecular engineering when fitted in the flow line of suitable material, excite the molecules and synthesis the material to yield hydrogen gas separated from the other constituents of the material or produce current carrying electrons for electrical output due to charge transfer, ionization and electrons movements from the material enabling it's usage for the production of clean energy.

### SUMMARY OF THE INVENTION

The present invention relates to molecular engineering of the materials enabling control of synthesis, catalysis, combustion, emissions from combustion and heat engine cycle performance. The present invention is also directed to a novel process and construction of a synthesizer for molecular engineering of the fluids and chemicals to control catalysis, combustion, emissions from combustion and the performance of any heat engine cycle or chemical process.

### DETAILED DESCRIPTION OF THE INVENTION

Accordingly, the present invention provides a process for the molecular engineering of a material using any one or all or a combination of the following namely the electro magnetic waves, microwaves, millimeter waves, ultrasonic waves, sound waves, surface waves, plasma waves, longitudinal electrostatic wiggler, space charge waves, electrostatic ion-cyclotron waves in the presence of an axial magnetic field to propagate through a wave guide containing the material and causing excitation of the electrons, acceleration of the electrons resulting in collisions or collision less space charge formation, plasma generation, plasma acceleration and plasma oscillations facilitating energy transfer with the waves and the plasma to take place for changing any one or all or a combinations of the following namely translational, rotational, vibrational energy level, electronic motions of the molecules of the material resulting in the changes in the molecular energies, change in bond angles, bond alternations, bond orientations, rearrangement of molecular structure, generation of pieco second radicals or particles in meta stable states depending upon the frequency, modes of propagation of the waves through the wave guide facilitating any one or all namely resonant capture of electrons, injection of electrons, hole transfer, energy catalysis, dark catalysis and changes in any one or all or a combination of the properties namely the viscosity, density, temperature, specific heat capacities, enthalpy, entropy, surface tension, activation energy level of the material which influence the chemical kinetics in a process and enhances the performance and overall efficiency of any machine or heat engine cycle.

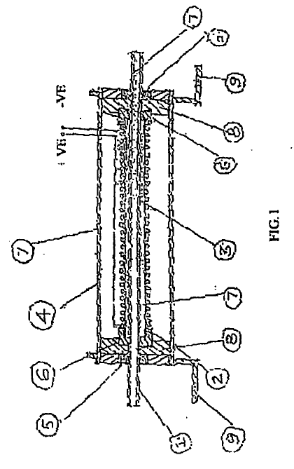

The present invention also provides a device/contrivance to facilitate the process of molecular engineering of a material as illustrated in the accompanied FIGS. 1-4, said device comprising:

a straight pipe (1) carries material, liquid, gas or sold;

the pipe (1) is enclosed by another bigger diameter pipe (2) with its ends closed by bushes (6) acting as supports for to support holding pipe (1) and also keeping both the pipes (1) & (2) on the same axis as their centers;

an insulated copper, aluminum or any conducting material with plurality of wire turns (3) for carrying current are wound over the pipe (2) and the pipe (2) is magnetized by applying an electric potential;

a co-axial system comprising pipe (1) and pipe (2) is enclosed by another pipe (4) with its ends closed by metal disk or plate having hole at the center similar to a washer with insulated bushes (5) and (8) for holding the co-axial pipe (1) and pipe (2) system also at its center ensuring that the center lines of all the three pipes are in the same axis and the whole system acts as a magnetron and as a cold plasma generator cum plasma accelerator;

The hollow cavity of pipe (2) with semiconductor layer (7) may contain air or any gas or any dielectric material or made vacuum filled with vacuum and the space between pipe (2) and cavity of pipe (4) with semiconductor layer (7) is filled with air at an atmospheric pressure or with any gas or with any dielectric insulation material or filed with vacuum;

the ends of the insulated wire turns are connected to an electric potential to pass electric current to magnetize the pipe (2) and to resonate electromagnetic waves inside the cavity of pipe (2) where the modes of propagation and frequency is dependent upon the diameter, length of the cavity pipe (2) for single cavity and dependent upon the diameters and lengths of pipe (2) and (1) for a co-axial cavity;

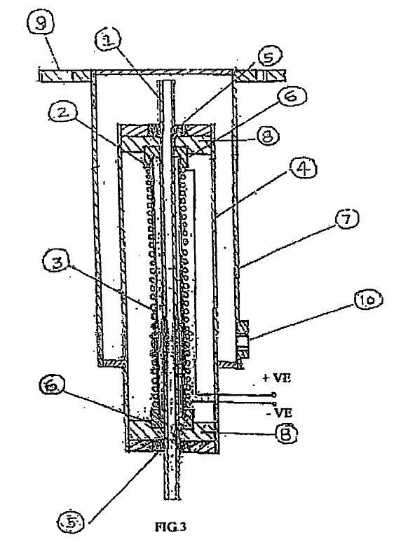

a casing (7) having a fluid flow passage (10) and serving as a means for cooling the device from over heating as also for increasing the temperature of fluid flowing through pipe (1) as shown in FIG. 3; and brackets (9) for earth grounding;

The functional features of the device of the present invention is further explained in the form of following embodiments:

The presence of pipe (1) in the same axis of pipe (2) make a co-axial cavity resonator and the frequency of electromagnetic waves or microwaves or any other waves generated and propagated through it depends upon the inner diameters of the pipe (1) and the inner diameter of pipe (2), the lengths of both pipe (1) and pipe (2).

Depending upon the material of pipe (1) and the dielectric property and material of bushes at the ends of pipe (2), the electric potential of pipe (1) and the magnetic and electrical behavior of pipe (1) could be altered other than pipe (2) or kept as the same as pipe (2). Pipe (1) also acts as a wave-guide for electromagnetic waves with various modes and other waves or oscillations generated.

When an electric potential is applied to the ends of the insulated wire turns and pipe (2) becomes magnetized, there is an axial field created by external coils and an azimuthal field created by internal conductors with a current value resulting in plasma contained between the concentric cylinders and the loss along field lines is prevented by an electric potential created by annular grids at the ends of the cylinder biased to negative potential by grounding to earth through brackets (9) or kept at positive potential as the case may be and electromagnetic waves starts resonating inside the cavities of pipe (4), pipe (2) pipe (1) due to co-axial construction or at least in any one of them, the electrons originating from the conduction band of the semiconductor layers (7) of the co-axial pipes due to thermal ionization, electromagnetic ionization of the semiconductor are excited, pipe (2) becomes a positive potential and pipe (1) too depending upon the end bush material and it's dielectric property and electrons are radially confined by the positive potential of the pipe (2) and pipe (1) and orbit around it in low angular momentum, non circular orbits and the electrons would be adiabatically compressed into the interaction region where the radial electric field would be large. This increase the electron density as well as the ratio of their perpendicular to parallel energy and electrons would rapidly transit the interaction region where they would resonantly transfer their energy to the radiation fields. This result in microwave amplification by stimulated emission radiation and an electron beam is produced whose distribution function is optimized for the power level requirements of the apparatus and the axis of the electron beam coincides with that of the wave guide pipe (1). Pipe (1) carries the liquid, gas or chemical or solid or all and when the material flows through pipe (1), the magnetic fields cause magneto hydrodynamic conditions along with excitation of the electrons in the conduction band of the semiconductor layer (7) of the pipe (1) and a plasma pinch is formed at the center line of flow with initialization of ionization and the microwaves or other waves act with the plasma pinch creating electron collisions and electron impact for further ionization with neo classical transport effect.

The properties of the material is altered under dynamic conditions or under steady state condition depending upon the material although for a detailed working of the device, a simple configuration with drawing is highlighted, the device could also be made of a straight pipe of any cross section other than hollow circular sections such as a hollow square section or hollow rectangular section or hollow elliptical section, hollow triangle section or hollow hexagon, pentagon or octagon sections or a combination of all hollow sections for varying the field characteristics and modes of propagation of electromagnetic waves and other types of waves and the cut off frequencies depending upon applications and end use.

In certain applications, the heat generated by the device due to microwave radiation, heating of the coil used for magnetization, heat released due to work done by electric fields to rotate polar molecules etc. could also be utilized by the liquid or gas or chemical by covering the major length of the outer pipe (4) by casing (7) having a fluid flow passage (10) and such arrangement serving as a means for cooling the device from over heating as also for increasing the temperature of fluid flowing through pipe (1) as shown in FIG. 3.

In certain applications, the wave guide is coupled to a tee joint at the input or at the output end or at both the ends facilitating injection of more than one material into the wave guide or for separation of any gaseous component formed during synthesis within the wave guide and the heat generated by the synthesizer used in conjunction with vacuum to cause fragmentation of certain fluids, hydrocarbons or chemicals to smaller compounds or elements.

It is abundantly clear from the foregoing description that the straight pipe (1) as material carrying media has multi various functions. It acts as a component of cavity resonator. It further acts as a wave-guide. Further the said straight pipe (1) acts as an applicator for transmission of microwaves and other forms and types of wave.

Another bigger diameter pipe (outer pipe) (2) and the enclosure pipe (4) acts as cavity resonator. The complete co-axial system with pipe (1), pipe (2) and pipe (4) having the same common axis represent a co-axial resonator for microwaves and any other waves for propagation and resonance and also function as a magnetron, as a cold plasma generator cum plasma accelerator.

The Pipe (1) as envisaged in the invention is either built into the device so as to be part of the synthesizer or a conventional pipe used in the conventional flow line can be readily substituted in place of aforesaid built in pipe (1). The structural arrangements may be incorporated in the synthesizer, which allows the possibilities of using the conventional flow lines for replacement of the pipe (1).

Though the embodiment of the invention as envisaged is described with respect to a material synthesizer, the invention with all its ramifications find acceptance in various areas of applications such as internal combustion engines, outer combustion equipments fluid flow lines, process flow lines of chemical equipments for heating the fluids or as an electronic catalytic convertor for chemical synthesis or in fuel flow lines as an electronic catalytic convertor or as a fuel reformulator and as a heat pump in any heat conversion equipments such as an internal combustion engine, combustion equipment, heat transfer equipments, refrigeration equipments and in the flow lines of exhaust gas of any combustion equipments or in the exhaust gas lines of internal combustion engines for further emission reduction or synthesis of burnt and un burnt chemicals or for excitation of the carbon di oxide, carbon monoxide and oxides of nitrogen molecules in the exhaust gases for synthesis and making the excited exhaust gas suitable for LASER beam generation or in the flow lines of suitable materials for synthesis and making the material suitable for generation or separation of hydrogen from it's constituents or for obtaining electrical output from the current carrying electron motions.

The device is installed before the engine carburetor, fuel injection system for altering the properties of the hydrocarbon fuel for better combustion efficiency, reduced fuel consumption and lesser emissions either as a separate device in the fluid flow lines or integrated as a component in the assembly of equipments.

This synthesizer is housed in an enclosure with an inlet and an outlet for material flow through it and provided with two wires for energizing it with an electric potential for initiating the novel process during the material flow through it. The device excites the molecules and the electrons of the materials contained within it to cause charge transfer necessary for the molecular engineering resulting in any one or all of the following namely change of the molecular structure, bond orientations, bond strength, physical property, chemical property. It has no moving parts and the device is fixed in the material flow line of the process or in the material flow line of a machine either at the input or at the output or in between the flow lines to obtain maximum benefit.

The device is fitted to any petrol, diesel, gas engines or systems using hydrocarbon fuels very easily without any alterations of the engine in minutes.

The device makes use of some of the properties of matter such as “Electro Negativity”, “Influence of lone Pairs of Atoms”, “Strains & Conformation” to change bonding angles of molecules, affinity of oxygen atoms and ionization and separation of Macro Molecules under MHD Influence, Hydrogenation due to Catalysis, dia and Paramagnetic Anisotropy of Macro molecules, magnetic orientation of molecules for altering the molecular properties including activation energy level of the hydrocarbon fuel just before mixing with air for ignition in the engine.

Petrol, Diesel, Kerosene, Lubricating Oil refined from crude oil belong to Alkanes group of hydrocarbons with different boiling points can be used as fuel. The physical and chemical properties of alkanes are a function first of the number of carbon atoms it contains and second of it's specific structure. The excitation of the fuel in the presence of a suitable catalyst cause Ring Strain and make the alkanes loose a hydrogen atom. The hydrogen atoms cause hydrgenolysis of the minute sulphur giving out hydrogen sulphide which again under excited conditions dissociate into hydrogen and sulphur. The hydrogen is absorbed in the metal surface of the catalyst or released as a gas while the sulphur is adsorbed on the metal and semiconductor surface layers of the wave guide and act as a catalyst accelerating the fuel reformulation. The H-H bond is weakened and a reactive hydrogen atom adds to the double bond or to Lone Pairs of atoms. The trace of moisture and additives in the fuels cause Electro negativity which under external field excitation make oxygen also bond with Lone Pairs of Atoms altering the molecular structure of the fuel, it's physical and chemical properties including Activation Energy Level, volatility, fuel quality. A lean mixture is a definite answer for reduced pollution and fuel economy. A lean mixture burns slowly and yields higher thermal efficiency due to ratio of specific heat approaching that of air and with less dissociation loss due to lower combustion temperature and lower losses to cooling water and air. However a lean mixture is very difficult to ignite and engines operating with lean mixture require high compression ratio or high-octane fuel. The field excitation cause rearrangement of molecular clusters and molecular bonding and the catalyst cause hetrogeneous catalysed gas phase combustion which is an efficient way of burning a lean mixture outside the flammability limits without forming pollutants. Gas phase ignition of hydrocarbon increase flame velocity and reduce minimum ignition energy. The catalytic surface temperature controls the reaction rate instead of the ambient temperature.

Emission of carbon monoxide, nitrogen oxides and hydrocarbon are related to air fuel ratio. In a vehicle fitted with a catalytic convertor, the air fuel ratio is around 15:1 or less for less emission whereas with the invented device, the air fuel ratio is above 16:1 to 22:1 depending upon engine condition which results in fuel economy and less emission.

The ability of the engine to start with a lean mixture (with increased air input) reduced fuel consumption and less emission, less engine knocking (smoothness of engine working), less engine temperature are indicative of the physical and chemical property changes of the fuel due to molecular structure change made by the invented device.

It is evident that the device according to invention makes use of various scientific principles as enumerated in the body of the specification. The precision of the device is an important criteria to augment the working results and to achieve optimum operating results. The embodiment of the invention illustrated in the drawing is to explain the active principles involved in the construction and the operation of the device and the combined or otherwise synergistic effect of various active principles and components in the said device.

The invention is further explained in the form of following Examples. However, the following examples should not be construed as limiting the scope of the invention.

### Application No.1-Automobile Engine Emission Control

In the automobiles or with any internal combustion engines, the synthesizer is used as a pre engine on board fuel reformulator, resulting in the following benefits

1. Mileage increase by 15% to 25% depending upon engine conditions.

2. Reduced emissions and Improved engine performance due to automatic decarbonising and removal of varnish deposits in engine.

3. Optimum performance approximately 3000 kilometers after installation due to decarbonising and elimination of most of the accumulated engine varnish in used vehicles.

4. Installation in minutes.

5. No additives to fuel or altering of engine.

6. Saves petroleum fuel for the user and foreign exchange for the nation.

7. Suitable for Two wheelers, three wheelers Autos, Cars, Tempos, Jeeps, Diesel engine, Diesel and petrol generator, LPG system, Fishing Trawlers, Agricultural diesel pump sets, Power Tillers and Tractors.

### APPLICATION NO.2-REFRIGERATION AND COOLING APPLICATION

In the Refrigeration systems the synthesizer is installed inline in the refrigerant flow to add additional heat energy for enhancing the refrigerant cycle performance for better cooling or for reducing the compressor energy demand for lowering the power consumption. In smaller cooling systems and portable cooling units, the refrigerant compressor is replaced with the synthesizer installed in it's place and the synthesizer could carry out the job of heating the refrigerant instead of the compressor by using microwave heat before expanding the gas and the cooling system will function with a non moving and non wearing synthesizer doing the function of a compressor consuming less power.

### Application No.3-Synthesis to Make it Suitable for Laser Beam Generation from Automobile Exhaust

Carbon di oxide, which is abundant in the automobile exhaust along with oxides of nitrogen and available continuously during engine working makes the synthesizer most ideal for use with the automobile exhaust gas for exciting the exhaust gas molecules for further emission reductions and also for facilitating synthesis for generation of carbon di oxide laser beam which could be used for navigating the vehicle, collision control, using the laser beam for inducing ignition, fuel synthesis, accurate measurement of vehicles moving in front and back etc.

For application as a laser beam generator, the center pipe (1) is removed in FIG. 3 and the engine exhaust flows through passage fluid (10) and through the end bush (6) center hole, the gas enters the cavity of pipe (2) wherein the carbon di oxide and nitrogen oxide molecules are excited by the electromagnetic waves resulting in population inversion and emission of light. The inside of the bushes (6) will be coated with reflective coatings to facilitate reflections of the beam within the cavity. The bush at one end of pipe (2) will have a concave reflective shape while the bush (6) at the opposite end will have converging lens shape for pointing the reflected beam out of the synthesizer.

### APPLICATION NO.4-PREVENTION OF FLUID FREEZING IN THE PIPE LINES IN EXTREME WEATHER CONDITIONS

In extreme cold weather conditions, fluid freezing within the pipes and pipe cracking due to flow getting chocked is common. To avoid uninterrupted fluid flow and to prevent fluid freezing, the synthesizer is installed in line and the microwave heat keep the fluid warm preventing fluid freezing including maintaining the fluid viscosity facilitating easier pumping without resistance build up in the flow line.

### APPLICATION NO.5: PREVENTION OF SCALE FORMATION IN PIPE LINES OF SUGAR SYRUPS, HARD WATER LINES

The ionization of molecules by the microwaves prevents the scale formation by hard water in the internal flow passages of hard water lines and also in the sugar syrup lines. This lessens the pressure required for pumping the liquids and also saves the heating costs.

### Application No.6-Hydrogen Rich Fuel Generation from Diesel, Gasoline, Methanol and Ethanol

The synthesizer excites the weak C-H bonds present in the hydrocarbons and many OH bonds and radicals through PLASMA process and produces hydrogen or hydrogen rich fuels for clean combustion and for reduced emissions.

### Application No.7-Inline or Onboard Fuel Quality Improvements

The microwaves and Plasma cause electrons migrations resulting in fuel molecules with re arranged structures, changed volume fractions as evidenced from gas chromotograph testing, bond alternations, density and viscosity variations resulting in improved fuel quality having enhanced octane or cetane values for the gasoline and diesel fuels respectively.

### Application No.8-New Pharmaceuticals and Chemicals

Under excited conditions, the free radicals and pieco second radicals liberated recombine to yield new pharmaceuticals and chemicals as the rate constant and kinetics of chemical reactions are altered by the charge transfer effects under excited conditions in many reactions.

### Application No.9-As a Heat Pump With Non Moving Parts Using Microwave Energy

The microwave heat absorbed by the material flowing through the synthesizer makes the synthesizer an ideal heat pump with no moving part for adding energy input to any thermodynamic cycles for enhancing the thermal efficiency. Example is use in refrigeration cycle with or without the compressor.

### BRIEF DESCRIPTION OF THE ACCOMPANIED DIAGRAMS

FIG. 1 shows in elevation cross sectional view of the Fluid Synthesizer.

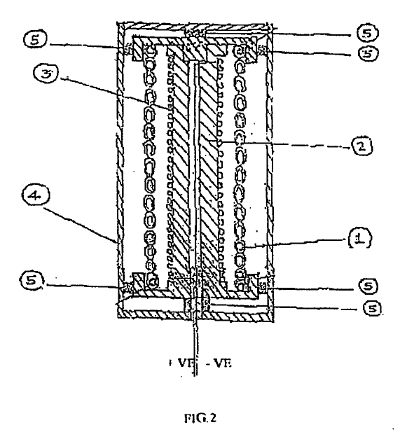

FIG. 2 shows in elevation the Fluid Synthesizer having tubular passage wound in the form of coil.

FIG. 3 shows in elevation of the Fluid Synthesizer having an enclosure over its outer surface in order to absorb the heat generated.

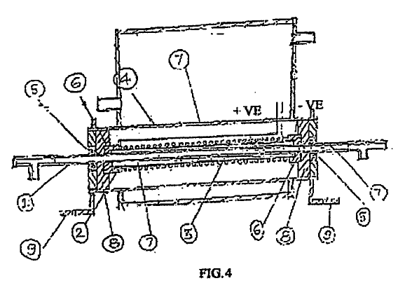

FIG. 4. shows in elevation the Fluid synthesizer within a vacuum chamber connected to an external vacuum source for reformulation, fragmentation and separation of low molecular structures.

### ADVANTAGES OF THE PRESENT INVENTION

1. The easy to install synthesizer when used with automobiles like two wheelers, three wheelers autos, cars, tempos, jeeps, diesel engine, diesel and petrol generators, LPG systems, fishing trawlers, agricultural diesel pump sets, power tillers and tractors, gives reduced emissions of carbon monoxide to almost near zero level, un burnt hydro carbon emissions reduced to few hundred ppm, diesel smoke reduction by over 60% from the initially measured value, enhanced mileage to the extent of about 15-25% depending upon engine conditions without any additions of additives to fuel or altering of engine and improves engine performance due to automatic decarbonising and removal of varnish deposits from engine combustion chamber.

2. Saves petroleum fuel for the user and foreign exchange for the nation.

3. When installed in the refrigeration fluid flow line, it enhances the efficiency of the cooling system and also saves compressor power and electrical energy.

4. When used with the automobile exhaust, facilitates excitation of carbon di oxide, carbon mono oxide and oxides of nitrogen molecules for synthesis making it suitable for laser beam generation which could be used for better navigation to avoid collision, pre treat fuel and also for ignition of the engine for controlled flame speed and enhanced combustion.

5. Molecular excitations yield hydrogen rich fuels in real time.

6. Accelerates the process of hydrogen generation from Methanol, Ethanol Diesel. Gasoline and many other liquids rich in hydrogen.

7. Facilitates newer pharmaceuticals due to radical formation during molecular excitations and changed kinetics of chemical processes.

8. To avoid fluid line freezing during winter in all liquid flow lines.

9. To prevent scale formation in hard water lines, syrup lines in the sugar factories.

10. Portable Antenna for mobile communications.

11. As a HEAT PUMP with no moving part for enhancing the efficiency of any heat power cycles or heat engines or refrigeration.

12. For changing the rate constants and kinetics of chemical reactions.

It will be understood that the foregoing description is only illustrative of the present invention and it is not intended that the practical applications of the invention be limited thereto to internal combustion engines, combustion technology only. Many other specific embodiments of the present invention and related practical applications will be apparent to one skilled in the art from the foregoing disclosure. All substitutions, alterations and modifications of the present invention, which come within the scope of the following claims, are to which the present invention is readily susceptible without departing from the spirit of the invention.

1. A unique process of molecular engineering of materials to control the synthesis, catalysis, combustion, emissions, heat engine cycle performance, kinetics of chemical reactions, physical and chemical properties of materials, waves generation, propagation and oscillations said process comprising the steps of:
(a). fabricating and using a synthesizer made of semiconductors, nanowires, nanotubes, co-axial cavity resonator cum plasma accelerator, wave guides, electrodes, electromagnetic source as described in FIG. 1-4;
(b). passing the desired material through the synthesizer;
(c). creating an axial electromagnetic field with coils energized by electric potential and an azimuthal field by the internal conductors of the synthesizer with a current value resulting in plasma within the synthesizer;
(d). the electromagnetic field along with or without heat and sound exciting the electrons in the conduction band of the semiconductors forming part of the arrays of the nanowires, nano tubes and in the semiconductor layers of the wave guide(s)/co-axial pipes, also the electrons of the material flowing through the wave guide facilitating arrays of the nanowires, nanotubes and the wide band gaps of the semiconductors to emit photons due to electron-hole plasma mechanism responsible for gain at room temperature resulting in laser action and the nanowires, nano tubes serving as fabry-perot cavities and a gain medium for light amplification;
(e) the said arrays of nanowires, nanotubes being formed by a process between the metal contacts of the co-axial conduits or wave guides or wave guide with electromagnetic source coil with appropriate number of turns over it by using semiconductor materials such as zinc oxide, silica, galliuim nitride, Ferric Oxide, fullerenes, vanadiumpentoxide and the like and filling the space between the coaxial conduits or wave guides containing the nanowire, nanotube arrays with any polymer or dielectric suitable for the specific nanowires, nanotubes and solidifying the polymer or dielectric using a catalyst and accelerator giving the composite material the desired combination of electric, optical, thermal and mechanical properties;
(f) the electromagnetic energy below the diffraction limit of the metal nanoparticle arrays cause resonances in the nano particles favoring plasmons, resulting in optical functionality in the nano scale resulting near fields coupling based on inter particle distance and the plasmon waves traveling within the wave guides;
(g). propagating, amplifying or attenuating the wave(s) in the wave guide(s) also causing magnetic circular dichroism;
(h). accelerating the electrons resulting in collisions or collision less ionization, space charge formation, cold plasma generation, plasma acceleration and plasma oscillations facilitating energy transfer;
(i). absorbtion or release of energy by the molecules due to wave interactions at the fermi surface and the energy gaps having width smaller than the ski layers retarding or facilitating the propagation of the electromagnetic waves resulting in the electrons density change due to charge transfer, an environment resembling electromagnetic nature of catalysis;
(j). release or absorption of energy being in the form of spectral luminescence or emission or radiation of microwaves or millimeter waves or surface waves or plasma waves or longitudinal electro static wiggler or space charge waves or electro static ion-cyclotron waves or alfen waves associated with the plasma or ultra sonic or sound waves or a combination of all above waves including electron waves;
(k). combining or filtering the released energies in the form of heat, light, spectral luminescence and multi various waves having different amplitudes and frequencies to propagate or resonate along with the electromagnetic waves generated due to the application of electric potential facilitating transfer of energy to take place with the waves the plasma and the material through electron resonant capture or electron capture or electron injection;
(l). facilitating synergistic effect of heat, light, spectral luminescence, multi various wave energies and the electrons movements to alter the translational energy, vibrational energy, rotational energy and electronic motions of molecules of the material for effecting changes to the molecular energy waves, change in molecular bond angles resulting in conformations or bond alternations for changing any one or all or a combinations of physical and chemical properties of the material;
(m). facilitating changes to any one or the combination of the following namely the temperature, density, viscosity, change of state, space charges in the material, pieco second radicals formation from the excitations of molecules, re arrangement of molecular bonds of the material activation energy levels, enthalpy, entropy, surface tension, specific heat capacities resulting in the material having an altered characteristics under excited conditions or upto few pieco/nono seconds after passing out of the synthesizer for changing the rate constant values for chemical reactions, catalysis, synthesis and the kinetics of reactions in a chemical process, combustion resulting in altered heat transfer, heat release, altered end products with altered mass balance.

2. The process according to claim 1, wherein the waves are selected from electro magnetic waves, microwaves, millimeter waves, ultrasonic waves, sound waves, surface waves, plasma waves, longitudinal electrostatic wiggler, space charge waves and electrostatic ion-cyclotron waves, Alfen waves associated with plasma, infra red heat wave, spectral luminescence.

3. The process according to claim 1, wherein the generated waves are either used as a single wave or in combination of desired waves or in combination of all the waves with complexity.

4. The process according to claim 1, wherein in step (1) of claim 1 the physical and chemical property changes that are effected include translational, rotational, vibrational energy levels, electronic motions of the molecules of the material resulting in the changes in the molecular energies, change in bond angles, bond alternations, bond orientations, re-arragement of molecular structure generation of pieco second radicals or particles depending upon the frequency, modes of propagation of the waves through the wave guide.

5. The process according to claim 1, wherein the said process facilitates “ENERGY CATALYSIS” in the material processed with the production of pieco second particles or electrons or atoms or molecules of meta stable states capable of transferring energy from the electron gas of the discharge plasma to the molecules to be activated through electron capture process or electron injection process or through resonant capture process in addition to conventional surface catalysis.

6. The process according to claim 1, wherein said process facilitates changes in any one or all or a combination of the properties of the material selected from viscosity, density, temperature, specific heat capacities, change of state, space changes in the material, enthalpy, entropy, surface tension, activation enegy level of the material which influence the chemical kinetics in a process that enhances the performance and overall efficiency of any machine or heat engine cycle.

7. A synthesizer as illustrated in FIGS. 1-4 to carry out the process of molecular engineering of a material(s) to control the synthesis catalysis, combustion, emissions and heat engine cycle performance when the processed material is used and the synthesizer functioning as waves generator oscillator, optical and plasmon wave guides, a solid state ionics device enabling cavity quantum electrodynamics, the said device comprising:
(a). a central open ended conduit (1) means for transporting material(s) and acting as an inlet and an outlet, wave guide and an applicator of microwave heat to the flowing material;
(b). hollow bigger diameter conduit (2) with its ends closed by bushes(6) enclosing and supporting the pipe(1);
(c). the conduits of steps (a) and (b) are positioned on the same axis as their centers;
(d). plurality of coils (3) are wound over the conduit (2) to make it an electromagnetic source with the application of an electric potential;
(e). the ends of the coils are connected to an electric potential to facilitate flow of current and to resonate electromagnetic waves inside the cavity of pipe (2) and pipe (1) and also to excite the dielectric composite with nonowire, nanotubes and semiconductors above the coil turns and in between the annulus of the coaxial wave guides cum pipes (2) and (4) through heating of the coil and also by the electron waves encircling the coil enabling plasma acceleration, emission of photons due to electron-hole plasma mechanism responsible for gain at room temperature resulting in maser and laser action and the nanowires, nanotubes serving as fabry-perot cavities and a gain medium for light amplification, wave propagation within wave guide cum pipe;
(f). a co-axial system comprising conduits (1) and (2) is enclosed by another conduit (4) with its ends closed by a metal disk having a means to hold pipes (1) and (2) at the center with insulated bushes (5) and (8);
(g). the cavity spaces between pipe (1) and pipe (2) is filled with air at atmospheric pressure or with any gas or with any dielectric composite with nanowires, nanotubes or made vacuum depending upon the output requirement of the synthesizer;
(h). the permitivity of the dielectric composite insulator is varied depending upon the frequency of waves used for interaction with the material being synthesized from full insulation to partial insulation having semiconductance by encapsulating it with any one or all namely silica nanowire, zinc oxide, nonowire, gallium nitride and ferric oxide nanowire and nanotubes arrays formed with a catalyst, accelerator and resin composite;
(i). a semiconductor layer coating (7) over the pipe(1), pipes (2) and (4) on the outer surfaces and inner surfaces;
(j). a fluid flow passage (10) enclosing pipe (4) as a means for cooling the synthesizer from over heating and also for increasing the temperature of fluid flow through pipe (1) whenever needed depending upon the material to be synthesized;
(k). brackets (9) on either side of the pipe (4) for earth grounding;
(l). Bushes (6) on either side of pipe (2) to make pipe (1) magnetizeable or non magnetizable and also to make it a positive potential(anode) or negative potential(cathode) element of the synthesizer depending upon the material and insulation characteristics;

8. The synthesizer according to claim 7, wherein the insulated coils are made of a conducting material selected from copper, aluminum or any conducting material or in the form of a circular printed circuit board over pipe (2) from one single length wire of same diameter or metal link of same continuity with one positive and one negative ends or wound over the pipe(2) with different lengths of wires of same diameter or metal link bring same continuity to form different coils each having one positive and one negative ends and the wire or metal link ends of each coil connected to the same electric potential from the same source or from different sources or to different electric potentials, the split coils formation facilitating:
(a) equal load sharing by the batteries when such batteries of same voltages are connected in series for other applications and charged with a common charger or alternator with an output voltage suitable for the batteries in series connection;
(b) connecting to direct current supply or alternating current supply or high frequency supply or combinations of various power supplies as inputs at the same time by connecting the different ends of each coil turn to each different power source at the same time for producing complex excitations and complex waves propagation;
(c). use of coil turns made of same diameter wires or different diameter wires over the pipe (2) for varying the impedance or resistance or characteristics of the synthesizer to suit different materials processing and for use as in steps (a) and (b) above or for connecting such ends with alternate polarities of the electric potential so as to change the poles of magnetism and consequently the waves of propagation depending upon such changes in the connections of the coils ends with the power sources terminals;
(d) inter connection of the ends of different diameter coil turns in series or in parallel for manipulation of the heating and waves generation, propagation and oscillations;

9. The synthesizer according to claim 7, wherein the center pipe (1) and the pipe (2) on which insulated wire turns are wound acts as a positive potential (anode), wave guide, co-axial resonator and the outer hollow tube(4) enclosing them and insulated with bush (5) acts as a co-axial resonator, wave guide, plasma generator cum accelerator enclosing the nanowires, nanotubes and the semiconductors within the annulus and having negative potential behaving as cathode or bias, a configuration in which the amplification, the modes of propagation, intensity and pattern of distribution of the magnetic and electric fields in the radial and longitudinal directions within the cavities and the frequency of the electromagnetic waves and other waves interacting with the liquid, gas or chemicals passing through the synthesizer, are dependent upon the parameters selected from
diameters of the circular hollow section of the inner and the outer pipes,
the lengths of the pipes,
the thickness of the pipes,
the geometry of the cross section of the pipes and their dimensions in case of non circular sections,
number of turns of insulated copper or current carrying wire and their diameter,
current and voltage through the insulated wire turns incase of direct current and additionally another parameter namely frequency in case of alternating current,
permitivity of the cavity medium and
permeability of the material of pipes.

10. The synthesizer according to claim 7, wherein the said synthesizer having an enclosure with fluid flow passages (10) for circulating the material over pipe (4) facilitating:
(a). absorption of the heat generated by the synthesizer enabling the material being processed to make use of the heat while also cooling the synthesizer
(b). use of the synthesizer for two different applications at the same time by suitably adding one more fluid flow passage (10 ) at the extreme ends of the enclosure (7) making the total number of fluid flow passages provided in the enclosure (7) as two in number and extending the pipe (1) to protrude outside the bracket (9) and the bracket provided on either ends of the pipe (4) to facilitate flow of one material through pipe(1) for the synthesis and flow of a different material through the fluid flow passages (10) to make use of the heat generated by the synthesizer by a different material.
(c). use of the synthesizer as a pre engine on board fuel reformulator cum electronic catalytic convertor for the fuel synthesis in a diesel or gasoline engine by passing the fuel through pipe (1) and allowing at the same time the freon liquid of the vehicle air conditioner to pass through the fluid flow passages (10) in the enclosure (7) so as to cool the synthesizer and also to provide the heat pump effect to the airconditioner to alter it's thermodynamic efficiency, performance, cooling effect and power absorbed.

11. The synthesizer according to claim 7, wherein the co-axial system acts as a magnetron, plasma generator cum accelerator enabling radial confinement of electrons when an electric potential is applied to the ends of the insulated coil copper or any conducting material wound on the pipe acting as positive potential or anode and the outer hollow pipe acting as negative potential or cathode resulting in an axial magnetic field with an azimuthal field by internal conductors with a current value resulting in plasma, the electrons orbit around it in low angular momentum non circular orbits with high frequency, adiabatically compressed into the interaction region where the radial electric field is large so that the adiabatic compression increases the electron density as well as their perpendicular to parallel energy facilitating resonant energy transfer to the radiation fields producing fast waves with rich harmonics resulting in microwave amplification by stimulated emission radiation.

12. The synthesizer according to claim 7, wherein the length of the conduit acting as a co-axial cavity resonator of hollow circular cross section is enclosed by another hollow circular cross section tube as enclosure cum wave guide for all or any one or a combination of the electro magnetic waves selected from microwaves, millimeter waves, ultrasonic waves, sound waves, surface waves, plasma waves, longitudinal electrostatic wiggler, space charge waves electrostatic ion-cyclotron waves, electron waves generation, amplification, propagation and interaction of these waves with liquid, gas or chemicals passing within it.

13. The synthesizer according to claim 7, wherein the co-axial system ensures that the centerlines of all the three pipes of the device are in the same axis and the whole system acts as a magnetron, a cold plasma generator cum accelerator, co-axial cavity antenna or wave guide and a solid state ionics device to control synthesis, catalysis, combustion, emissions, heat engine cycle performance, kinetics of chemical reactions, physical and chemical properties of materials, waves generation propagation and oscillations

14. The synthesizer according to claim 7, wherein the said synthesizer acting as a magnetron for microwave generation, a co-axial cavity resonator, plasma generator cum accelerator, wave guide and as an applicator for transmitting the microwave and it's heat to the flowing material also used as a portable wave generator for communications and constructed from conduits(pipes) of any geometry sections other than circular section including a combination of several geometry cross section pipes, ionizes the material by energy transfer from the electromagnetic waves or other waves at a resonant frequency or at near resonant frequency characteristics of the material passed through the synthesizer.

15. The synthesizer according to claim 7, wherein said synthesizer having a center tube for flow of material at any moment through it and the flow of the material taking place in the presence of catalyst or without catalyst and the semiconductor layer within the cavity may or may not be acting as a catalyst depending upon the material processed.

16. The synthesizer according to claim 7, wherein the said synthesizer having a center tube for flow of material and built into the device so as to be part of the synthesizer is replaced by a conventional pipe used in the conventional flow line retaining the structure arrangements incorporated in the synthesizer allowing the possibilities of using the conventional flow line itself as a part of the co-axial system, wave guide and an applicator and the remaining structural arrangement inserted into an existing conventional pipe like carrying the material.

17. The synthesizer according to claim 7, wherein the said synthesizer is flexibly designed for the control of an automotive exhaust emissions and is positioned optimally at any of the desired locations selected from:
either before the carburetor or within the carburetor,
before the fuel injector, within the injector or after the injector,
before the fuel injection pump,
in the fuel intake manifold and
anywhere before engine fuel intake or in the fuel return line, fuel lines connecting the engine and the fuel tank or within the fuel tank itself.

18. The synthesizer according to claim 7, wherein the said synthesizer when used for exhaust emission control, and fitted as a pre engine device in the fuel line of the engine or vehicle alters the fuel properties by partial ionizing, releasing hydrogen ions or gas, the released hydrogen reacting with the traces of sulphur in the fuels forming hydrogen sulphide at room temperature or at excited conditions and corresponding temperatures enabling the face (100) of the iron in the applicator to fix the sulphur atoms on quaternary sites giving the structure C (2×9) causing the basic square lattice to be turned to 45 degrees in relation to the lattice of the substrate, the surface concentration of the adsorption increasing with several passes of the fuel making the structure C(22×2),C(18×2),C(16×2),C(14×2) and finally C(10×2), a similar adsorption also occurring with the ionized nickel atoms of the applicator with the formation of P(2×2) turning to root 3×root 3 rotated by 30 degrees making sulphur atoms located in ternary sites until a compact arrangement of sulphur atoms with a coincidence lattice P(5×5) in relation to the atom of the substrate takes place and the changed structure, the adsorbed layers formed by the minute sulphur traces in the fuels also acting as a surface catalyst and oxidizer under the excited conditions for accelerating the synthesis and the synthesized fuels changing the kinetics of combustion, reactions, altering the mass balance of products of combustion for reduced un-burnt fuel and toxic gas emissions enabling enhanced engine operation with a lesser fuel to air ratio mixture possible due to change in the molecular energy levels and heat value of the fuel change in vapor versus air ratio of fuel, activation energy of the fuel.

19. The synthesizer according to claim 7, wherein the said synthesizer is used as an Electronic Catalytic Convertor or an onboard Fuel Reformulator for making the fuel hydrogen rich in real time in the vehicle /engine or as a Common Catalytic Convertor for any automotive fuels selected from diesel, leaded and unleaded petrol, blended fuels with ethanol or any other additives, gaseous fuels and liquid fuels for improving their quality in real time.

20. The synthesizer according to claim 7, wherein the same synthesizer acts as a microwave generator, a co-axial cavity resonator, plasma accelerator, a wave guide for electromagnetic waves, surface waves, sound waves, optical waves, plasmon waves and many other waves created by the solid state ionics construction and cavity quantum electrodynamics, an applicator of microwave heat to the material(s) and the microwaves and lift emissions from the semiconductors and nanowires, nanotubes are amplified by stimulated emission radiation action within the synthesizer and the axis of the electron beam coinciding with that of the wave guide and the electrons movement radially confined by the electric field between the positive potential center conductor and negative potential outer conductor of the device,
(a). enabling the electrons moving in axis encircling orbits with a substantial fraction of their kinetic energy transverse to the axis and the microwaves or other waves, and the photon emissions from the semiconductors or laser beams from the nanowires, nanotubes causing the synthesis;
(b). the synthesis involves all or any one namely partial or full ionization of liquid, gas or chemical releasing ions, free radicals pieco second particles, change in bond angle orientation of the molecules, molecules with rearranged structure and bond alter nation, oxidation, reduction, absorption, hydrogenolysis, heat release or heat absorption, organic synthesis resulting in addition, hydration, breaking of bonds, coupling, re-arrangement, fragmentation depending upon the molecular structure of the fluid handled, change in the viscosity, density, temperature, activation energy, surface tension and other physical, chemical characteristics of the material

21. The synthesizer according to claim 7, wherein the said synthesizer is also used as an electronic inline heat pump in the refrigerant flow line of refrigeration machine along with the compressor or as a replacement for the compressor of smaller capacity systems or used as a heat pump in any heat engine cycles for enhancing the thermodynamic performance.

22. The synthesizer according to claim 7, wherein the said synthesizer is also used for the generation or separation of hydrogen from any hydrogen rich fluids or material as a fuel reformulator for the production of clean fuel for use in a fuel cell or used as a power cell producing electrical potential by making use of the electrons generated by the excitations and during the magneto hydrodynamic flow conditions

23. The synthesizer according to claim 7, wherein the said synthesizer is used for the excitation of automobile or any combustion engine exhaust gases containing carbon di oxide, carbon monoxide and oxides of nitrogen for further processing of emission reductions, generation of LASER beam using the exhaust carbon di oxide along with other accessories for further use of the carbondioxide laser beam for applications including precise vehicle navigation for collision prevention, laser beam ignition of fuel, laser beam fuel synthesis etc.

24. The synthesizer according to claim 7, wherein said synthesizer is used in conjunction with any vacuum system or along with injection of additional materials or without injection of additional materials and used to synthesize or fragment any fluid or chemical to lighter atoms or molecules or used for the production of new formulations under the excited conditions of energy catalysis including new pharmaceuticals and chemicals.

25. The synthesizer according to claim 7, wherein the said synthesizer is used for prevention of scale formation in water carrying pipes, sugar syrup pipes, water softening and similar applications including use as a inline heater in the fluid lines during winter to prevent fluid freezing in the flow lines and to change the viscosity of fluid facilitating easier pumping with reduced energy or power.

26. The synthesizer according to claim 7, wherein the same device by executing the same process and used in multi various application to control the synthesis, catalysis, combustion, emissions, heat engine cycle performance, kinetics of chemical reaction, physical and chemical properties of materials and used in the fuel cells, waves generator and oscillator for communications, refrigeration, line heating, scale formation removal, water softening, laser beam production from automobile exhaust or any combustion engine exhaust for further applications, production of new pharmaceuticals or chemicals formulations or materials.

27. The synthesizer according to claim 7, wherein an inner conductor cum wave guide is seized for propagation of electromagnetic waves in a pre selected frequency range with a plurality of outer conductors cum wave guides generally positioned co-axial with the inner conductor cum wave guide, each successive outer conductors cum wave guides having diameter larger than the adjacent outer conductor cum wave guide, one of the plurality of the outer conductors cum wave guide positioned with respect to the inner conductor cum wave guide to form a cavity between the inner conductor cum wave guide and the adjacent outer conductor cum wave guide, each successive pair of outer conductors cum wave guide positioned to form a cavity, each cavity seized for propagating electromagnetic waves in the pre selected frequency range when dielectric gases or air is used in the cavities and waves of different frequencies generated and propagated under different modes making the synthesizer to act as a co-axial antenna or resonator for communications.

28. The synthesizer according to claim 7, wherein the ends of the coils are connected to an electric potential to facilitate flow of current and to resonate electromagnetic waves inside the co-axial cavities resulting in the excitation of the dielectric composite with nanowires, nanotubes and semiconductors above the coil turns and in between the annulus of the co-axial wave guides cum pipes along with heating of the coil the electron waves encircling the coil enabling plasma acceleration, emission of photons due to electron-hole plasma mechanism responsible for gain at room temperature resulting in maser or laser action and the nanowires, nanotubes serving as fabry-perot cavities and a guide medium for light amplification, wave propagation and oscillations within wave guide cum pipes making it an unique solid state ionics device, optical wave guide, plasmon wave guide, a cavity quantum dynamic device;

29. The synthesizer according to claim 7, wherein the generated microwave heat is used for vaporizing gasoline or diesel and converting the liquid fuels into gaseous state for feeding any engines or vehicles directly or in combination with other gaseous fuels for reduced emissions and enhanced performance.

30. The synthesizer according to claim 7, wherein the generated electromagnetic radiation and microwaves are used for accelerated drying of food products, grains and irradiation at economical costs and also for preventing icing of the automobile radiator water or engine cooling paths in the extreme weather conditions including use in many general heating applications.

31. The synthesizer according to claim 7, wherein the said synthesizer in conjunction with any vacuum system as in FIG. (4) enables the microwave heat generated by it to vaporize sea water or saline water or contaminated water at reduced temperatures facilitating pure water production when used alone or along with solar heating systems and also as a stand by heating source with any solar heating or cooling system or output performance compensator in the event of fluctuations in the solar energy insolation or during non sun shine hours such as night or rainy days.

32. The synthesizer according to claim 7, wherein the said synthesizer is used as an electronic inline heat pump in addition to existing heat source(s) or as an independent heating source in a vapor absorption refrigeration cycle flow line for enhancing the thermodynamic performance.

33. The synthesizer according to claim 7, wherein the said synthesizer with co-axial wave guides propagating microwaves and other waves at different modes with electric and magnetic field components confined within the cavities, the electric fields within the wave guide cavities used to alter the flame characteristics such as flame color, soot formation, flame temperature, flame length within the cavities including extinguishing the flame with an appropriate electric field.
